(19) United States
(12) Patent Application Publication
(10) Pub. No.: US 2008/0255670 A1
(43) Pub. Date: Oct. 16, 2008

(54) PROSTHETIC OR ORTHOTIC JOINT

(75) Inventors: Herman Boiten, Gottingen (DE); Jens Northemann, Duderstadt (DE)

Correspondence Address: FAEGRE & BENSON LLP;PATENT DOCKETING, 2200 WELLS FARGO CENTER, 90 SOUTH SEVENTH STREET, MINNEAPOLIS, MN, 55402-3901, US

(73) Assignee: Otto Bock Healthcare IP GmbH & Co. KG, Duderstadt (DE)

(21) Appl. No.: 12/059,905

(22) Filed: Mar. 31, 2008

(30) Foreign Application Priority Data
Mar. 29, 2007 (DE) 102007015560.5

(51) Int. Cl. A61F 2/30

(52) U.S. Cl. 623/18.11

(57) ABSTRACT

Prosthetic or orthotic joint with at least two joint segments that are mounted rotatably to each other around a joint axis, and a pivotal piston mounted in torque-proof fashion in one of the upper or lower joint segments. The pivotal piston is received in a displacement chamber containing a fluid, with the pivotal piston subdividing the displacement chamber into two partial chambers that are connected with each other via a connecting channel at least across a selected pivoting angle range of the pivotal piston. At least one flow section determining contour is formed between the pivotal piston and one selected displacement chamber wall, which is in a fluidic connection with the connecting channel and the displacement chamber across a selected rotation angle. The contour also provides a varying free flow section for the passage of the fluid dependent on the rotational position of the pivotal piston.

### CROSS-REFERENCE TO RELATED APPLICATION

This application claims priority to German application 10 2007 015 560.5, filed Mar. 29, 1007, entitled PROSTHESEN-ODER ORTHESENGELENK, which is herein incorporated by reference in its entirety.

### TECHNICAL FIELD

The invention relates to a prosthetic or orthotic joint with first and second joint segments that are mounted rotatably to each other around a joint axis, and a pivotal piston that is arranged on the first or second joint segments and is guided in a displacement chamber containing a fluid.

### BACKGROUND

The invention is particularly well suited for control of the movements of a knee joint or of a foot joint. But in principle, other areas of application in orthotics and prosthetics are conceivable, for example in the case of hip joint prostheses or orthoses.

From the prior art, so-called rotation hydraulics are known in which the joint parts of a prosthesis or orthosis are rotationally connected with each other and a pivotal piston subdivides a displacement chamber into two partial chambers. The partial chambers are connected with each other via two throttle non-return valves connected in parallel and acting in opposite directions.

In this context, changes of the flow section of the connecting channel can be accomplished from the outside via an axially movable throttle rod so that the fluid, in particular hydraulic oil, can flow during a flexion or extension movement from one partial chamber into the other partial chamber, but at a higher flow resistance. Such a joint mechanism is described in DE 43 38 946 C1. A change of the section of the connecting channel can be accomplished as a function of an axial force that occurs, for example, when one shifts one's weight to one foot. This requires an axially movable mounting of a throttle rod.

Conventional rotation hydraulic systems have a damping effect that is constant. In the event of a higher energy supply into the damping device, for example as a consequence of a faster pace, a larger flexion or extension will result. The desired flexion angle or extension angle will therefore be exceeded.

### SUMMARY

The objective of the invention at hand is to provide a prosthesis or orthosis joint with which an adjustable damping of the rotation movement can be achieved in a simple manner.

A prosthetic or orthotic joint in accordance with the invention has at least two joint parts or segments, such as upper and lower parts, that are mounted rotatably to each other around a joint axis. A pivotal piston is arranged in a torque-proof fashion on the upper or lower parts of the joint and is guided in a displacement chamber containing a fluid. The pivotal piston subdivides the displacement chamber into two partial chambers that are connected with each other at least across a connecting channel that is a selected deviation angle range of the pivoting piston. Between the pivoting piston and the displacement chamber at least one flow section determining contour is formed that is connected in fluidic fashion with the connecting channel and the displacement chamber via a selected, set or predetermined rotation angle, which, dependent on the angle position of the pivoting piston, provides a varying free flow section for the passage of the fluid.

The prosthesis joint or, respectively, the orthosis joint in accordance with the invention makes it possible to alter the fluid or oil flow during the movement of the joint parts relative to each other, for example the upper part relative to the lower part of the joint. Dependent on the rotational position of the joint, i.e. during the movement, a fluid stream from one partial chamber into the other partial chamber is provided which is modified depending on the position of the joint and thus of the pivotal piston. By changing the free flow section for the fluid through the contour between the pivotal piston and the displacement chamber walls, for example through a channel in the pivotal piston and/or in the displacement chamber, it is possible to provide angle-dependent damping in a simple fashion. As a result, a rotation hydraulic system is realized within the joint that provides higher damping at a certain rotational position.

A further development of the invention provides that between the displacement chamber and the pivotal piston two contours are formed or, respectively, provided that are allocated to the respective junctions of the connecting channel. As a result, a fluid stream can flow into the partial chamber in the direction of the extension as well as in the direction of the flexion via the junctions of the connecting channel and the contour within the pivotal piston or the displacement chamber. This makes it possible to provide flexion-angle dependent, changeable damping effects both in the extension direction and in the flexion direction, since the free flow section for the passage of the fluid is changed separately. Varying contours are provided for extension and flexion in order to provide varying damping effects that are variable across the angle range of the rotation. As a result, a pivoting angle dependent and pivoting direction dependent change of the damping effect can be provided by building in the contours or channels into the pivoting piston or into the housing that forms the displacement chamber.

One embodiment of the invention provides for the contour or the channel to be built into one axial plane of the pivotal piston. In the case of several contours or channels, it is provided that they are built into the axial plane of the pivotal piston separately from each other, for example by milling or grinding the contour or contours or, respectively, the channel or channels into the axial plane of the pivotal piston, so that a basic form of a damping curve can be determined in a simple manner. The axial plane of the pivotal piston is the surface that is not designed as a piston plate and that is essentially formed as a circular cylinder. The axial plane slides on a correspondingly formed contour of the displacement chamber and seals the partial chambers against each other on the side of the pivotal piston lying opposite the piston plate. Alternatively or supplementarily to a contour in the axial plane, the latter may also be formed in the displacement chamber or, more precisely, in the wall of the housing forming the displacement chamber and allocated to the axial plane. As a result, a basic damping effect, for example, is provided by the contour in the displacement chamber that is modified by the installation of an adjustable pivotal piston that is adjusted to the requirement of the user of the prosthesis or of the orthosis.

In one variant of the invention, the contour may overlap, to a varying degree, the junction of the connecting channel depending on the rotational position of the pivoting piston, i.e., it may reduce or prevent the flow into the contour by partially or completely closing the junction. In that case, the contour will not overlap the junction during the entire rotation movement during which a fluidic connection exists between the connecting channel and the contour. Instead, a limited cross section of the junction is made to overlap the flow channel formed by the contour, thereby providing a change of the free flow section at the interface between the contour and the junction. In the case of a radially aligned junction and a contour arranged or formed in the direction of the circumference, the changing overlap is achieved by an axial relocation of the contour. In the case of an axial arrangement of the junction, the change is produced by a corresponding spiral shaped arrangement and formation of the contour or of the channel for a partial overlapping and change of the effective flow section.

One further development provides for the contour or the channel to be made to completely overlap the junction for a predetermined rotation angle range. The contour or channel bases are then at different distances to the junction dependent on the rotational position of the pivotal piston. The greater the distance between the contour or channel base and the junction, the greater the free flow section. If the contour base is moved very close to the junction, the flow section decreases in the transition area, such that only a small amount of fluid can pass this bottle neck which will lead to an increased damping effect. The advantage of such an arrangement, in the case of a radially aligned junction, lies in the fact that the channel or the contour may be formed in a straight line; only the depth of the milling of the contour or of the channel will be changed in order to make different damping rates available. Such a design may also be combined with a channel contour or a channel course that will alter the cross section of the section and the degree of overlapping of the junction.

In principle, it is possible that several junctions of the connecting channel with the partial chambers are present; in particular, two junctions may be present in each partial chamber through which the fluid can flow from one partial chamber through the connecting channel into the second partial chamber. The additional junctions may be arranged in a series or parallel to the junctions connected to the contour and/or contours or the channel and/or channels in order to be able to provide a coordinated damping effect.

For the adjustment of the damping effect, it is provided that at least one non- return valve is arranged in the connecting channel. Preferably, several non-return valves are arranged for flexion and extension that do not interfere with each other, making it possible to provide a corresponding control of the damping effect for both directions of movement.

In addition, there is the possibility that at least one control valve is built into the connecting channel in order to generate high damping values across the entire range of movements, particularly in flexion and/or bending directions, which are generated independently of the contour and the damping effect of the channels in the pivotal piston. The damping may be canceled almost completely by means of the control valve in order to achieve a minimal damping effect that is independent of the rotational position, for example when bicycling.

In order to be able to preset a basic damping level, at least one adjustable throttle is built into the connecting channel. This throttle is preferably adjustable from outside of the joint, in order to be able to perform an adjustment of the prosthesis or orthosis joint by the respective joint user.

With an arrangement of the contours or channels in the axial plane of the pivotal piston, the path or, respectively, the angle adjustability is limited by the length of the sealing surface. In the case of rotation hydraulics, this is the surface that rests directly on the axis of rotation. If the desired pivoting angle at which the damping effect is to be varied doesn't create a connection between the two partial chambers, the desired path or, respectively, the pivoting angle can be achieved through a parallel arrangement of several channels. In the case of a parallel arrangement, the first channel will then be shorter than the length of the sealing surface between the axial plane and the junction.

### BRIEF DESCRIPTION OF THE DRAWINGS

FIG. 1 is a lateral view of a prosthesis joint in accordance with the present invention.

FIG. 2 is a sectional view of a prosthesis joint, shown in FIG. 1.

FIG. 3 is a partial view of a prosthesis joint in accordance with the present invention in a schematic presentation.

FIG. 4 is an individual presentation of a pivotal piston in accordance with the present invention.

FIG. 4a is a variant of FIG. 4.

FIG. 5 is a lateral view of a built-in pivotal piston with a hydraulic circuit diagram in accordance with the present invention.

FIG. 6 shows a flow course of the built-in pivotal piston of FIG. 5 at the start of flexion.

FIG. 7 shows a flow course of the piston of FIG. 5 with progressed flexion.

FIG. 8 shows a flow course at a position in accordance with FIG. 7 in extension direction.

FIG. 9 shows a position at the end of flexion in accordance with FIGS. 5-8.

FIG. 10 shows a switch position of the piston of FIGS. 5-9 during the standing phase.

FIG. 11 shows an example of a damping course in accordance with the present invention.

### DETAILED DESCRIPTION

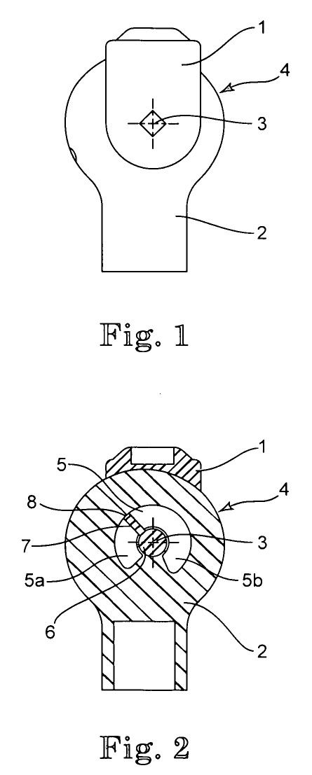

In FIG. 1, a prosthesis joint in accordance with the present invention is shown in a lateral view, in this case a prosthetic knee joint. The prosthetic knee joint has two joint parts or segments 1, 2, namely one upper joint part 1 and one lower joint part 2, which are connected with each other rotatably around an axis 3. In principle, embodiments with more than two joint parts 1, 2 are possible and provided. In the following, for reasons of clarity, an upper joint part and a lower joint part 1, 2 will be mentioned. Connecting parts not shown in detail are provided on the upper joint part 1 as well as on the lower joint part 2 by means of which, on the one hand, the prosthetic joint can be attached to the prosthetic user and, on the other hand, other prosthetic devices can be attached to it. In the embodiment form shown, the upper joint part 1 is supported by a cylindrical surface 4 of the lower joint part 2. Due to an eccentric arrangement of the axis 3 in the lower joint part 2, the upper joint part 1 runs to a stop on the surface 4 of the lower joint part 2, meaning that a separate joint block may be dispensed with.

In FIG. 2, the prosthetic knee joint is shown in a sectional presentation. A displacement chamber 5, in which a pivotal piston 6 is mounted in rotatable fashion, is formed in the lower joint part 2. The pivotal piston 6 is mounted in torque-proof fashion on the upper joint part 1 and is rotated together with the upper joint part 1 relative to the lower joint part 2 when the joint is moved. In principle, a reverse arrangement is possible as well. The pivotal piston 6 pivots around the rotational axis 3 and has an essentially cylindrical base body and a piston plate 7 attached to it, which extends radially away from the rotational axis 3 to the edge of the displacement chamber 5. A sealing strip 8 is arranged at the extreme radial end of the piston plate 7. The piston plate 7 divides the displacement chamber 5 into two partial chambers 5a, 5b; the sealing strip 8 seals the two partial chambers 5a, 5b from each other. Also, a sealing element may abut the base body 6 in order to achieve the most complete sealing effect possible between the two partial chambers 5a, 5b to the effect that no leaking flow occurs between the partial chambers 5a, 5b. In order to be able to displace the upper joint part 1 with respect to the lower joint part 2 in the first place, a connecting channel 9 is provided between the partial chambers 5a and 5b, as will be shown and explained later. Without the connecting channel 9, a displacement of the pivotal piston 6 would only occur by fluid transport due to leak flow, which would practically mean a blockage of the joint.

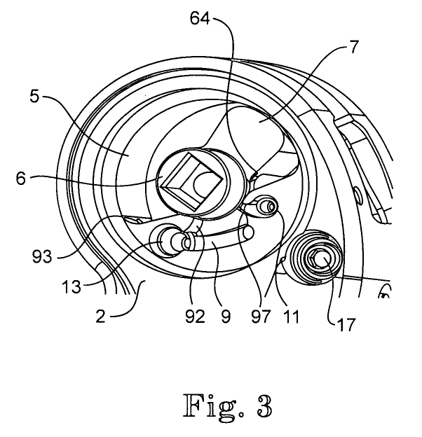

FIG. 3 shows a partial presentation of the prosthetic or orthotic joint without the upper joint part 1 in a partially assembled presentation. The lower joint part 2 is shown together with the displacement chamber 5 and the pivotal piston 6, together with the piston plate 7, mounted in it in rotatable fashion. Non-return valves 11, 13 are arranged in the lower joint part 2 which prevent a displacement of the joint in the direction of an extension but which do permit a flexion. A connecting channel 9 is provided in the lower joint part 2 through which fluid can flow from the displacement chamber 5, such that a material flow of the fluid can flow along between the partial chambers 5a, 5b. Moreover, a flexion valve 17 can be seen which will be explained later.

Analogously to a possible flexion, it is provided that corresponding valves or throttles are present for an extension which also create a flowing connection of the two partial chambers 5a, 5b via the connecting channel 9.

An axial surface 63 within the pivotal piston 6 is the surface of the base body that is essentially formed cylindrically and which slides along a corresponding receptacle of the lower joint part 2. On one axial surface 63, contours in the form of channels are built in, of which one flexion channel 64 can be seen in FIG. 3. The connecting channel 9 branches out several times and forms several junctions 91, 92, 93 in the housing wall of the displacement chamber 5 connecting it with the displacement chamber 5 and pivotal piston 6; not all junctions can be seen in FIG. 3.

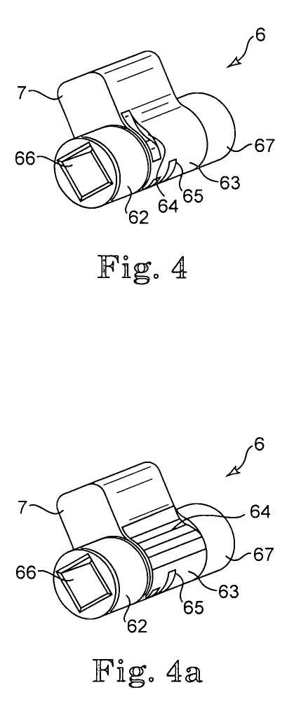

In FIG. 4, a pivotal piston 6 is shown in an individual presentation. The axial surface 63 with the built-in channels 64, 65 can be seen next to the piston plate 7. The axial surface 63 lies between two pegs 61, 62 on which the pivotal piston 6 is mounted within the lower joint part 2. Rectangular recesses 66 are formed on the front side of the pivotal piston 6 in order to fix the upper joint part 1 to the pivotal piston 6 in form-fitting and torque-proof fashion.

The channels 64, 65 are built into the radial circumference of the axial surface 63, preferably through milling, with the channels 64, 65 being designed straight in the embodiment shown, such that over the entire angular distance during which the channels 64, 65 lie opposite the respective allocated junctions 91, 92, they overlap the latter completely. As can be seen by way of the flexion channel 64, channel 64 has varying depths through which the free flow section is changed which are dependent on the rotational position of the pivotal piston 6 and thus of the upper joint part 1. In the area of the end of channel 64 facing away from the piston plate 7, the depth of the channel 64 approaches the axial surface 63 asymptotically and slowly tapers off, such that, from a predetermined rotational position which results from the length of the channel 64, the junction 92 is closed by the axial surface 63 which overlaps it in flush fashion. The flexion channel 64 does not extend across the entire circumference of the pivotal piston or, respectively, of the axial surface 63, but only across a minor part of the circumference, such that a fluidic connection of the connecting channel 9 with the corresponding partial chamber 5a, 5b is provided only for a partial section of the rotation. Alternatively, or supplementarily to a straight design of the channels, the latter may also be provided with an incline and extend spirally along the base body or, respectively, within the axial surface 63 in order to achieve a specific narrowing of the section of the junctions 91, 92.

In FIG. 4, the axially offset extension channel 65 can be seen as well which does not have a constant depth, such that the free flow section is variable and dependent on the rotational position. Here, too, the extension channel 65 tapers off into the axial surface 63 at the end of the extension channel 65 to the effect that from a certain rotational position on, the corresponding junction 91 is sealed off by the axial surface 63.

A variant of the embodiment of the pivotal body 6 is shown in FIG. 4a, in which the entire axial length of the axial surface 63 is provided with a flow section determining contour 64 so that one can no longer speak of a channel in the strict sense. A correspondingly wide contour 65 can be formed in the direction of the extension as well in order to provide variable damping effects depending on the pivotal angle and direction. If in the following a channel is mentioned, it will generally mean a flow section determining contour whose width may also extend across the entire length of the axial surface 63.

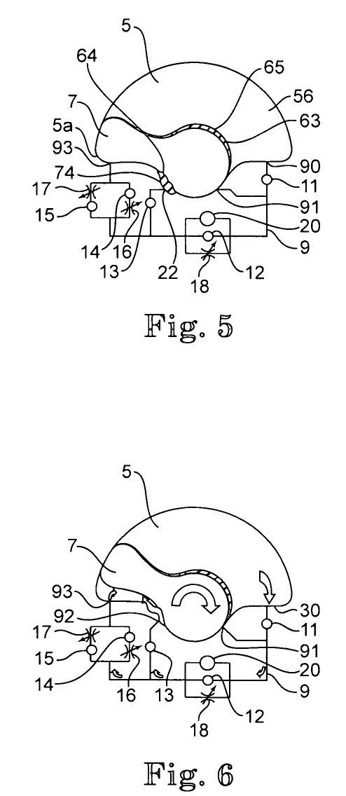

A hydraulic circuit and the arrangement of additional valves or throttles is shown in FIGS. 5 through 10. The connecting channel 9 with its junctions 90, 91, 92, 93 with the displacement chamber 5 connects the two partial chambers 5a and 5b with each other. In FIG. 5, it can be seen that the flexion channel 64 is shorter than the extension channel 65 and has a different contour, meaning that varying throttle contours are provided. The contours of channels 64, 65 make it possible to determine a basic form of a damping curve of the rotational hydraulic system within the prosthetic joint, which can be set or adjusted by further measures, for example by raising or lowering the basic damping level. In FIG. 5, it can also be seen that a flow section determining contour 74 is formed in the displacement chamber that lies opposite the flexion channel 64. This contour 74 can be designed as a channel or extend across the entire axial length of the displacement chamber. It is also possible that in the displacement chamber 5, or more precisely in the area of the displacement chamber 5 opposite the axial surface 63, a channel or a contour is formed opposite the extension channel 65 or the extension contour which is not shown in FIG. 5.

In the embodiment shown in accordance with FIG. 5, the prosthetic joint is in an extension position. The piston plate 7 minimizes the volume of the extension chamber 5a while the volume of the flexion chamber 5b is maximized. The connecting channel 9 between extension chamber 5a and flexion chamber 5b as partial chambers of the displacement chamber 5 branches out to such that two junctions 90, 91 open out into the flexion chamber 5b while junctions 92, 93 open out into the extension chamber 5a. The connecting channel 9 leads, via a non-return valve 11, from junction 90 to a stance phase valve 20 that is switched parallel to a non-return valve 12 and a standing phase throttle valve 18. Following the first non-return valve 11, a feed channel opens out into the connecting channel 9 from junction 91, which is allocated to the extension channel 65.

After the stance phase valve 20, the connecting channel 9 leads to a non-return valve 13 which blocks an extension movement. Parallel to that, a valve arrangement is arranged having parallel switched, counter directionally oriented non-return valves 14, 15 and, in each case, serially switched flexion and extension valves 17, 16. The two channels open out into junctions 92, 93.

The junctions 90, 91, 92, 94 do not necessarily have to be arranged at the walls of the displacement chamber 5 opposite the piston plate 7. It is also possible that the junctions 90, 93 are formed on the lateral, bent surface that lies opposite the distal end of the piston plate, such that the junctions 90, 93 are closed by the pivotal piston 6 from a selected position on, or that it is connected with the displacement chamber 5 via a channel that is not shown.

The start of the flexion movement is shown in FIG. 6. The contour 74 in the displacement chamber 5 is no longer shown in FIGS. 6 through 10; likewise, a corresponding extension contour is not shown, either. In principle, the following statements also apply to a combination of the channels 64, 65 or, respectively, contours 64, 65 together with the allocated channels 74 or contours in the displacement chamber housing. Starting from the extension position in accordance with FIG. 5, during a flexion, the fluid, in particular the hydraulic fluid, is initially guided through the always open junction 90 into the connecting channel 9 through the non-return valve 11. At that time, the second junction which is allocated to the extension channel 65 is still closed off by the axial surface 63. From the non-return valve 11, the fluid flows through the switching valve 20 which makes it possible to create a maximized high damping effect in the direction of the flexion, which functions independently of the contours of the channels 64, 65, 74 and, therefore, independently of the free flow sections of the channels 64, 65, 74 across the entire range of motions of the pivotal piston 6 and thus of the joint direction. After the switching valve 20, the fluid flows almost unhindered through the non-return valve 13, the junction 92 and the flexion channel 64 and the contour 74 into the extension chamber 5a. An additional partial stream flows through the non-return valve 15 and an adjustable flexion valve 17 through the second junction 93 into the extension chamber 5a. At the start of the flexion, the fluid therefore runs parallel through two channels, once via the contour through channel 64 and the contour 74 on the pivotal piston 6 and, in addition, through the flexion throttle valve 17. A very easy initiation of the swing phase is achieved through the additional opening and providing of a flow section in the junction 92, since the fluid can flow, almost without any resistance, through the non-return valve 13 into extension chamber 5a.

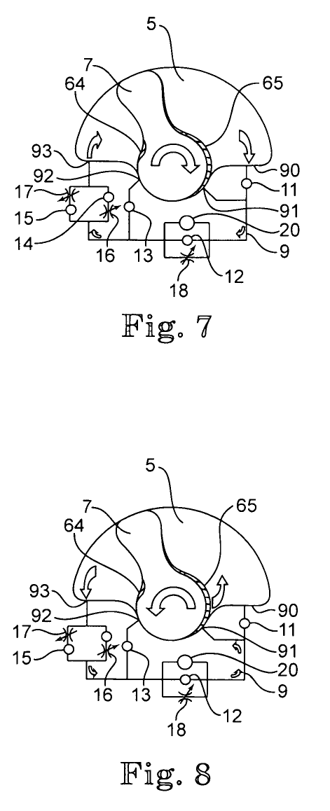

During an additional rotation of the pivotal piston 6, the axial surface 63 seals off the junction 92, as shown in FIG. 7. The fluid stream can therefore occur only via the stance phase valve 20 and the non-return valve 15, as well as the preset flexion valve 17. Due to the decreased overall flow section, the flexion damping increases as compared with the start of the rotation since a higher flow resistance is provided. In the interim area, between the start of the flexion and the blocking of the junction 92, a variation of the damping effect can be created by means of the contour of the flexion channel 64 and/or through the contour 74.

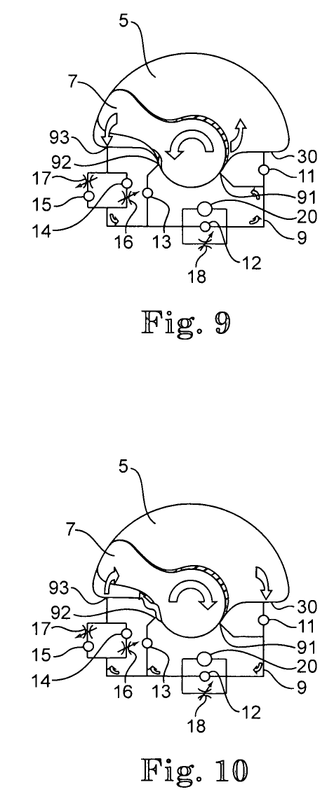

The damping during the extension movement is shown in FIGS. 8 and 9. The minimal damping during the initial phase of the extension is determined by the throttle valve 16. Through the throttle valve 16, the fluid flows through the non-return valve 12 via the branching of the connecting channel 9 through the junction 91 and the extension channel 65 into the flexion chamber 5b. The course of the damping effect can be set together with the throttle valve 16 and the contour of the extension channel 65. The level of the damping effect can be determined through the adjustable throttle valve 16, as well as through the contour of the channel 65 integrated on the axial surface 63. The throttle valve 16, as well as the channel 65, are arranged in series to each other.

At the end of the extension, as indicated in FIG. 9, the flow section decreases continually by means of a flattening of the extension channel 65, such that the junction 91 is closed successively. The slow narrowing of the free flow section up to a complete closure provides an increasing damping effect which makes it possible that a hard final end stop is prevented, even in the case of unexpectedly high forces. The fluid stream through the narrowing contour of the channel 65 is noticeably reduced towards the end of the extension movement, which is represented by a thinner line.

FIG. 10 shows a stance phase position of the prosthetic knee joint during which the stance phase valve 20, which can be designed as a switching valve, is closed. The closing of the stance phase valve 20 creates a high pressure for the throttle valve 18. After the stance phase throttle valve 18, the fluid is distributed across the available connections up to the junctions 90, 91. However, the resistance created thereby is noticeably lower than at the stance phase throttle valve 18, such that the influence of the contours of the channels 64, 65 is negligibly low and may be disregarded.

All throttle valves 16, 17, 18 may be designed to be presettable in order to allow for adjustment by the respective prosthetic or orthotic user, and the prevailing operating conditions. Preferably, the throttle valves 16, 17, 18 are adjustable from outside the connecting channel 9, making an easy adjustment possible at any time.

The fluid flow and thus the damping effect of the prosthetic or orthotic joint during the flexion, as well as during the extension, is determined or at least partially determined by the contour of the channels 64, 65 located in the pivotal piston 6. As a result, no elaborate motorized adjustment or a movable mounting of separate controlling devices are required. In principle, it is possible and provided that the contours 64, 65, 74 or the channels are designed as adjustable, for example via rotating bushings or adjustment elements that can alter the contour in axial or radial directions. In spite of the adjustability, if an adjustment has been done, the relevant rotation range or rotation angle in which the free flow section is influenced remains fixed.

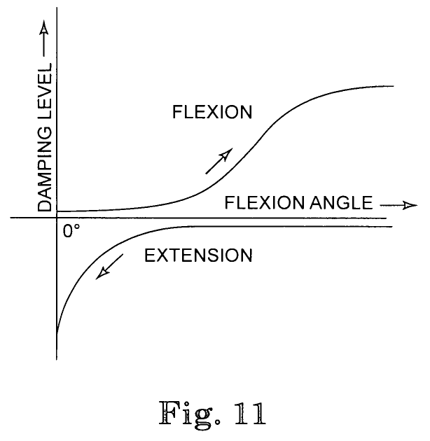

An exemplary path of the damping level that is dependent on the flexion angle is shown in FIG. 11. The top curve shows the path of the damping effect during flexion, i.e. bending, while the bottom curve shows the damping path during extension, i.e. stretching. The path of the curves corresponds to the form of contours 64, 65 in the pivotal piston 6, in accordance with FIGS. 6 through 10. During flexion, the bending of the joint, e.g. of the knee joint, is made possible with an initial relatively minor damping effect, with the fluid being able to flow essentially unhindered through the connecting channel 9 and the two junctions 92, 93. From a pre-determined flexion angle on, the junction 92 is closed off by the axial surface, thereby increasing the damping effect across the flexion angle. The further damping then occurs via the flexion throttle valve 17 which sets a maximum value.

Conversely, during an extension across almost the entire range, the fluid can flow through the junction 91 into the partial chamber 5b, thereby providing a low resistance due to a low damping effect. Only shortly before the end of the extension, i.e., in the proximity of the extension stop, the axial surface 63 increasingly closes the junction 91. The increase in the damping effect is provided through a continual narrowing of the flow section through contour 65 until the junction 91 is completely closed. Following a complete closure of the junction 91, a fluid flow occurs only by way of leakage flows. The basic damping effect of the extension is set via the extension throttle valve 14 in conjunction with the contour 65.

The following is claimed:

1. A prosthetic or orthotic joint comprising:
first and second joint segments mounted rotatably to each other around a joint axis;
a pivotal piston mounted in torque-proof fashion to one of the upper or lower joint segments;
a displacement chamber provided within the other one of the upper or lower joint segments, the displacement chamber containing a fluid and receiving the pivotal piston within the chamber, the displacement chamber subdivided by the pivotal piston into two partial chambers;
a connecting channel connecting the two partial chambers to each other at least across a selected pivoting angle sector of the pivotal piston; and
at least one flow section determining contour formed between the pivotal piston and one selected displacement chamber wall, the contour in fluidic connection with the connecting channel and the displacement chamber across a selected rotation angle, such that the contour provides a varying free flow section for the passage of the fluid which is dependent on the rotational position of the pivotal piston.

2. The prosthetic or orthotic joint of claim 1, wherein the at least one contour comprises two contours.

3. The prosthetic or orthotic joint of claim 2, wherein one of the two contours is configured for extension and the other contour is configured differently for flexion.

4. The prosthetic or orthotic joint of claim 1, wherein the contour is provided in an axial surface of the pivotal piston and/or in one displacement chamber wall.

5. The prosthetic or orthotic joint of claim 1, further comprising a junction between the connecting channel, the pivotal piston and the partial chambers, and wherein the contour overlaps the junction to a varying degree that is dependent on the rotational position of the pivotal piston.

6. The prosthetic or orthotic joint of claim 5, wherein the contour comprises a base and wherein the base is at varying distances from the junction which are dependent on the rotational position of the pivotal piston.

7. The prosthetic or orthotic joint of claim 5, further comprising additional junctions between the connecting channels, pivotal piston and the partial chambers.

8. The prosthetic or orthotic joint of claim 1, further comprising at least one non-return valve arranged in the connecting channel.

9. The prosthetic or orthotic joint of claim 1, further comprising at least one switching valve arranged in the connecting channel.

10. The prosthetic or orthotic joint of claim 9, wherein the switching valve operates across the entire range of movement of the pivotal piston independently of the contour.

11. The prosthetic or orthotic joint of claim 1, further comprising at least one adjustable throttle arranged in the connecting channel.

12. The prosthetic or orthotic joint of claims 11, wherein the throttle is adjustable from outside of the connecting channel.

13. The prosthetic or orthotic joint of claim 1, wherein the contour is configured as a channel.
